(19) United States
(12) Patent Application Publication
(10) Pub. No.: US 2004/0087999 A1
(43) Pub. Date: May 6, 2004

(54) Vascular filter with improved anchor or other position retention

(76) Inventors: Gjalt Bosma, Opeinde (NL); Ruldolf T. Mulder, Groningen (NL)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/285,218

(22) Filed: Oct. 31, 2002

(51) Int. Cl. A61M 29/00

(52) U.S. Cl. 606/200

(57) ABSTRACT

The present invention relates to a vascular filter which can be placed in a blood vessel, for the purpose of intercepting thrombus, and which has improved structural features for enhancing position stability at the desired site. These features may include improved barbs or anchor designs. An example of an improved anchor for retaining a vascular filter in position includes for example cutting a barb member from a circumferential side of a filter member or rib, or bending part of a barb slightly in an outward direction to form a gentle spring, or a bevel or bend of an outwardly extending barb toward the longitudinal direction. The various features of the present invention may be used singly or in any combination, as desired in a particular vascular filter.

### BACKGROUND AND SUMMARY OF THE INVENTION

1. Technical Background

The present invention relates to a vascular filter which can be temporarily or permanently placed inside a blood vessel for the purpose of intercepting thrombus.

2. Discussion

Some basic types of vascular filters are generally known, wherein a single filter element, mesh or member extends across the direction of flow inside a blood vessel. Several features are desirable for vascular filters, including non-surgical or percutaneous delivery of the filter to a desired site, and expansion from a preferably small initial size to an expanded working size that matches the vascular anatomy at the desired site. Also, a vascular filter should of course preferably capture a sufficient percentage of thrombus, while allowing blood to flow freely through the filter.

Another desirable feature is a capability to remain reliably in the desired position in a patient's anatomy, referred to as “position retention.” One easy attempt at position retention is to wedge a vascular filter against the blood vessel wall by sizing it with a dimension slightly larger than the inside diameter of the blood vessel. In addition, a vascular filter should preferably have a design whereby the filter is self-centering and stable in the vessel, such that the filter has little or no tendency to “tilt” which might thereafter result in less effective capturing of thrombus. Some vascular filters may be used in the vena cava, and may be described in such event as a “vena cava filter.”

Prior vascular filters may consist of a network of interconnected members or ribs in an umbrella or cone shape, which extend substantially in a radial direction in relation to the blood vessel. Unfortunately, an entire filter may shift position, especially in the unlikely event that one of the ribs might break. In addition, the free ends of the ribs, which may be positioned under a certain pressure against the internal wall of the blood vessel, may cause trauma to the vessel wall, or may become embedded within.

Another possible design is essentially a screen placed across a transverse dimension of a vessel. Again, it is desirable to take measures to resist “tilting” by this screen type of filter.

A disadvantage of known vascular filters may be this possibility of shifting position or tilting inside the blood vessel, even when the filter maintains its proper shape, if a prior vascular filter may have been incorrectly placed in a blood vessel which is too wide. In such an event, a vascular filter may not grab sufficient hold on the internal wall of the blood vessel.

Accordingly, an object of the present invention is to provide a vascular filter for delivery through a catheter in a compressed shape, which tends to resiliently expand within a blood vessel and to retain the desired position and orientation. The vascular filter tends to trap thrombus or particles, and resist their movement further downstream. The filter includes, in a position of use, an outer shape corresponding to the internal diameter of the blood vessel, and one or more filter elements transverse to the longitudinal direction of the vessel.

It may also be desirable to provide a vascular filter which can be implanted temporarily, rather than permanently. To help in successful retrieval, a factor is to avoid “endothelialization” or in-growth of the vessel wall and tissue around the structural members of the filter. On a temporary retrievable filter, it is also desirable to provide releasable temporary position stabilizers, to resist the tilting possibility described above and to enhance position retention.

Some vascular filters provide anchors or small barbs for improving position retention, which extend in radial directions outward from the ribs. One successful design which is sufficient for a variety of applications is shown in FIG. 4, having barbs cut out from a central section of the filter member. The barbs tend to gently hold the filter in place inside the blood vessel.

The known configuration of the barbs shown in FIG. 4 provides a first and second longitudinal cut 38 and 40 formed in one or more longitudinal members of the filter, as well as at least one angled cut 42 connecting the two longitudinal cuts 38 and 40. The resulting pattern provides a barb 32 that tends to lie flat with the rest of the filter during its insertion in a compressed shape, and which tends to extend outward slightly when the filter expands.

In contrast, a design having some of the features along the lines of the present invention is depicted in FIG. 5 and 6. One of the possible design features includes barbs that are cut out from a side portion of the rib. This improved anchor feature presents one rather than two longitudinal cuts in a filter rib, which may tend to strengthen the filter at the location of the anchor.

A vascular filter along the lines of the present invention provides several advantages, including strengthening the entire filter, and reducing the level of stress in each rib at the location of the barbs.

Also, vascular filters of the present invention that are retrievable, rather than permanent filters, may provide additional advantages. When a vascular filter is retrieved from a blood vessel, the entire filter is resiliently compressed to a relatively small diameter, for extraction through a catheter. When a retrievable vascular filter of the present invention is compressed for removal, the advanced design for the anchors or barbs enables the barb to gently dislodge from any tissue that may be present. In other words, the improved anchor design tends not to catch tissue between the barb member and the rib, because the rib is present only on one side of the barb member.

A vascular filter according to the present invention has a central tubular section that tends to resiliently exert slight outward pressure along a large section of contact area on the blood vessel wall. Accordingly, the filter tends to exert some small amount of pressure on the internal wall of the blood vessel, and tends to hold itself in place. The vascular filter will consequently tend not to shift position.

The term “tubular” is used in its broadest sense, to encompass any structure arranged a radial distance around a longitudinal axis. Accordingly, tubular includes any structure that (i) is cylindrical or not, such as for example an elliptical or polygonal cross-section, or any other regular or irregular cross-section; (ii) has a different or changing cross-section along its length; (iii) is arranged around a straight, curving, bent or discontinuous longitudinal axis; (iv) has an imperforate surface, or a periodic or other perforate, irregular or gapped surface or cross-section; (v) is spaced uniformly or irregularly, including being spaced varying radial distances from the longitudinal axis; (vi) has any desired combination of length or cross-sectional size.

In the preferred embodiment, a vascular filter according to the present invention includes a first and second filter section, arranged on either side of a body section. The body section and the filter sections thus enclose a space. Due to the elongated shape of the vascular filter according to the present invention, and the arranging of the first and second filter sections on either side of the body member, the present filter has an enhanced filtering effect. In other words, two opportunities have been created for intercepting thrombus moving inside the blood vessel.

In addition, because of this elongated shape the vascular filter according to the present invention tends to center itself within the lumen and not to rotate transversely or tilt over, which is another advantage of the present invention. Because the filter is preferably longitudinally symmetrical, the position of the filter inside the vena cava or another the blood vessel is therefore not dependent on the route along which it has been introduced. As a result of which, the physician has more freedom when choosing a route for introducing the vascular filter.

In a preferred embodiment, a vascular filter according to the present invention may preferably be formed out of one single piece, which provides advantages including simplicity.

Another preferred feature of the vena cava filter according to the present invention is that, when viewed along the longitudinal axis of the vessel, the filter sections have the shape of a regular polygon, and provides several smaller filtering “cells”. The purpose of these filtering cells is to intercept thrombus moving inside the blood vessel, and the smaller filtering cells tend to capture more thrombus. It is also preferable that the cells all be of the same size, to provide uniform filtering effect.

The filter sections, as arranged according to an embodiment described above on either side of the tubular body section, are preferably identical in shape, thereby enhancing the simplicity of the vascular filter according to the present invention.

Another possible benefit of the present invention relates to endothelialization, which is the healing of the vessel inner surface by endothelial cells. It is desirable to preserve these endothelial cells when removing a retrievable vascular filter, and the improved designs of the present invention tend to minimize any impact during retrieval.

It is of course possible to build various vascular filters according to the present invention, by various techniques and of various materials to obtain the desired features.

It should be noted that the present invention also relates to methods for manufacturing vascular filters as described herein.

These and various other objects, advantages and features of the invention will become apparent from the following description and claims, when considered in conjunction with the appended drawings. The invention will be explained in greater detail below with reference to the attached drawings of a number of examples of embodiments of the present invention.

### BRIEF DESCRIPTION OF THE DRAWINGS

FIG. 1 shows a perspective view of a vascular filter and delivery system in a position of use, and also illustrates schematically a manner in which a vascular filter may be placed inside a blood vessel;

FIG. 2 illustrates a side elevational view of a vascular filter according to the present invention, in an expanded configuration;

FIG. 3 shows an end view of the vascular filter of FIG. 2;

FIG. 4 shows a partial view of a vascular filter having a known anchor or barb design; and

FIGS. 5-11 show partial views of vascular filters having improved anchors or barbs according to the present invention.

### DETAILED DESCRIPTION OF THE PREFERRED EMBODIMENTS

The following description of the preferred embodiments of the present invention is merely illustrative in nature, and as such it does not limit in any way the present invention, its application, or uses. Numerous modifications may be made by those skilled in the art without departing from the true spirit and scope of the invention.

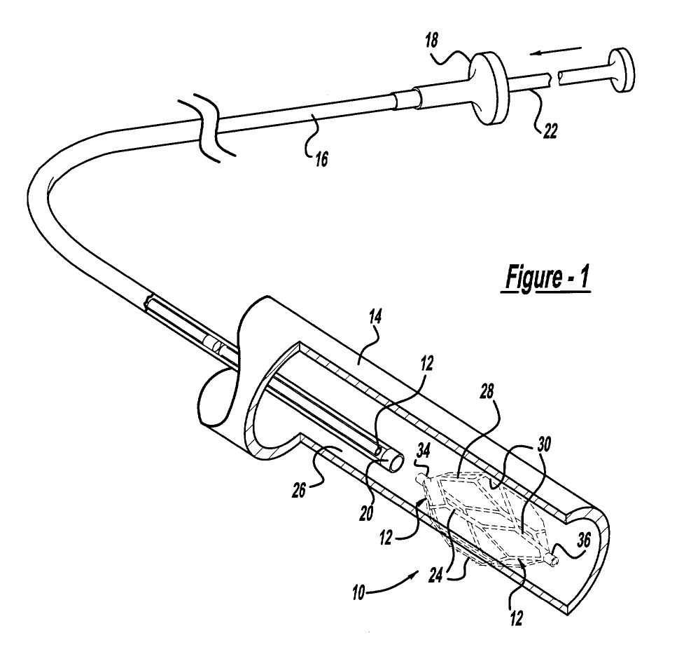

In FIG. 1 a vascular filter delivery system 10 along the lines of the present invention has been shown. In the situation illustrated in FIG. 1, a vascular filter 12 has been introduced into a blood vessel 14 through a catheter 16 which defines a lumen extending from a proximal end having a hub 18 to a distal opening 20. At least one vascular filter 12 is preferably arranged, in a compressed state, in the distal end of the catheter. The filter 12 is then pushed out the catheter distal opening 20 by a flexible pushing wire 22.

In an alternative embodiment, it is also possible that the filter 12 may be inserted at the proximal end of the catheter 16, and then pushed along the entire length of the catheter 16 by the pushing wire 22, after the catheter distal end 20 has been advanced to the desired position.

In any event, when the vascular filter 12 is ejected by the pushing wire 22 out from the distal tip 20 of the catheter 16 into the blood vessel 14, the vascular filter 12 will tend to resiliently expand after being released from the catheter 16. The material and design of the filter 12 result in resilient expansive forces that tend to cause the vascular filter 12 to take the illustrated shape.

The vascular filter 12 embodiment illustrated here may of course be used in the vena cava or any other desired site for treatment. The filter 12 preferably includes a number of ribs 24 extending in an axial or longitudinal direction along the internal wall 26 of the blood vessel 14. These ribs 24 together form an elongated central body section. On the proximal and distal ends of the ribs 24, a first and second filter section 28 and 30 have been arranged, with each section 28 and 30 forming a grid shape. Liquid inside the blood vessel 14 can pass through the vascular filter 12, but thrombus or particulates tend to be intercepted by one of the two filter sections 28 or 30.

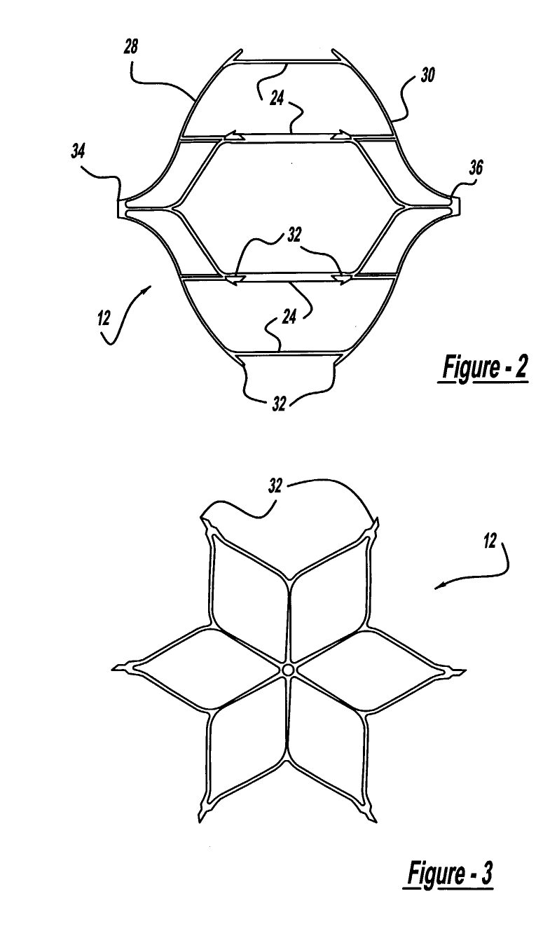

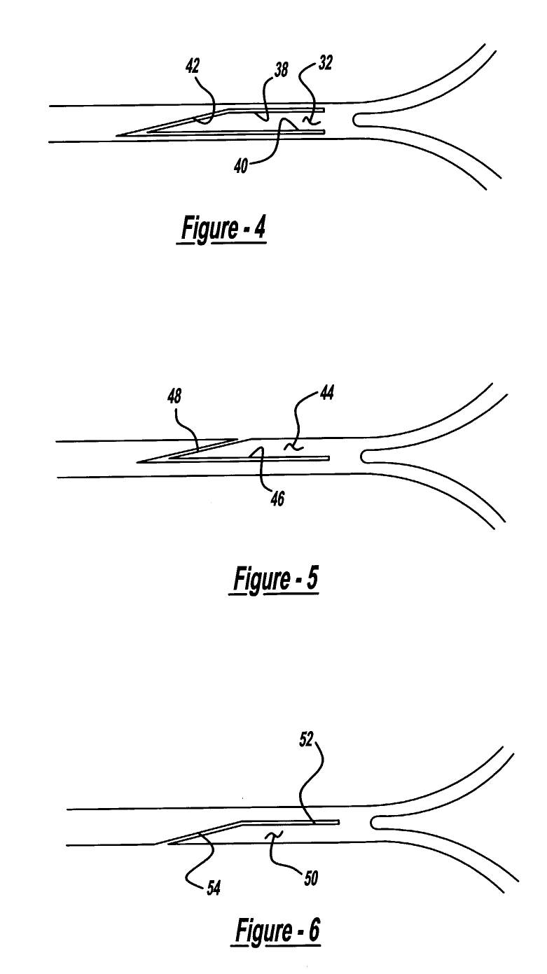

As shown in FIGS. 2 and 3, the filter 12 preferably has one or more barbs or anchors 32, located on one or more of the longitudinal ribs 24. The anchors 32 are preferably positioned at one or both ends of the longitudinal ribs 24, and may be directed in the proximal or distal directions. As shown in FIG. 2, opposing sets of proximal and distal anchors 32 may be arranged to face in distal and proximal directions respectively. This opposing arrangement causes the anchors 32 to resist movement of the filter 12 in both proximal and distal directions.

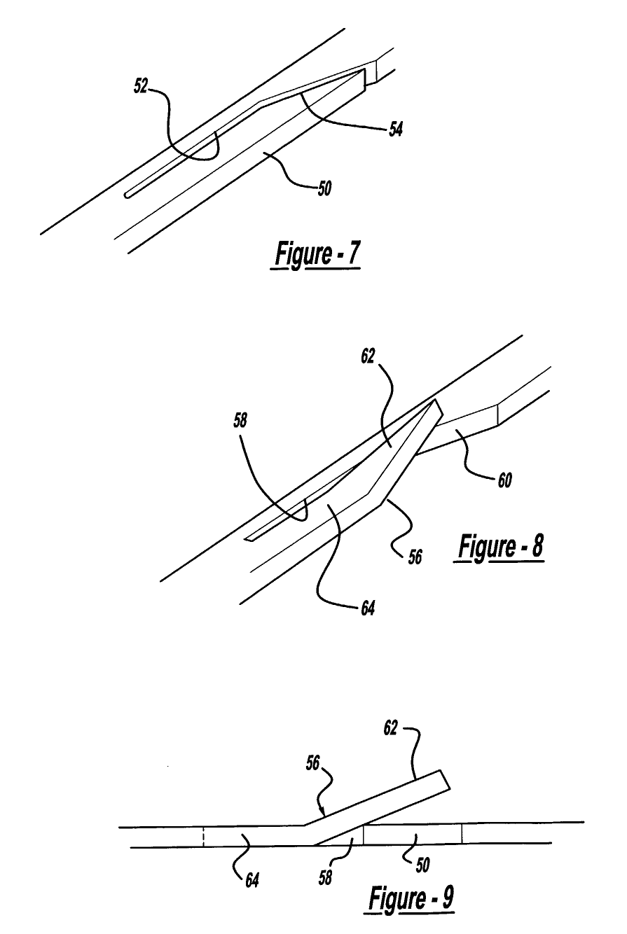

An example of conventional barbs or anchors 32 is depicted in FIG. 4. The filter is shown in a deployed or expanded state, so the barb 32 itself of the actual three-dimensional filter would stick up out of the page of FIG. 4. Barb 32 is defined by a first and second longitudinal cut 38 and 40 of different lengths, joined by an angled cut 42.

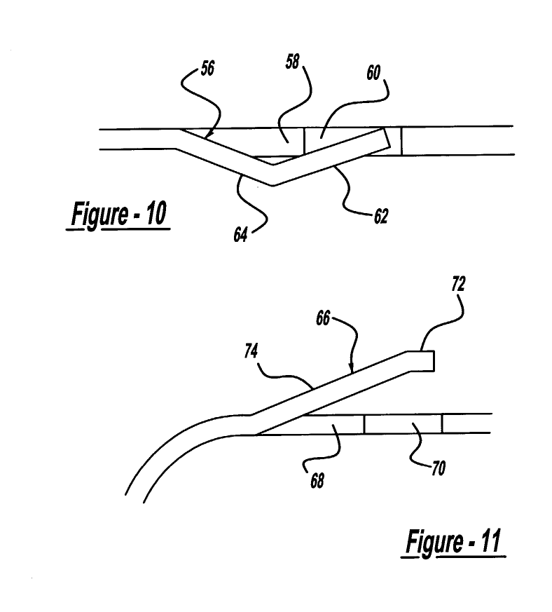

An embodiment of the present invention is shown in FIG. 5, in which an improved anchor 44 is defined by a single longitudinal cut 46 and a single angled cut 48, which extends from the longitudinal cut 46 to a circumferential edge of a rib of the filter. The longitudinal cut 46 and angled cut 48 form an acute angle.

An advantage of the present novel filter design include more effectively anchoring a retrievable filter in position, and another benefit is enabling easier retrieval of the filter. Some advantages of this design include that the strength and structure of the filter and the rib in which the anchor 44 is cut are more optimal. In addition, if the filter is retrievable and if any tissue may be present between the anchor 44 and the filter rib, the improved anchor of the present invention reduces any chance that may be possible of catching such tissue, thereby enhancing removal of the filter from the vessel.

Another embodiment of the present invention is shown in FIGS. 6 and 7, in which an improved anchor 50 is defined by a single longitudinal cut 52 and a single angled cut 54, which extends from the longitudinal cut 52 to a circumferential edge of a rib of the filter. The longitudinal cut 52 and angled cut 54 form an obtuse angle.

Another embodiment of the present invention would include an anchor in which the angle between a longitudinal cut and an angled cut equals a right angle, or 90 degrees. Such a design would provide an even more gentle position retention feature.

Another embodiment of the present invention is shown in FIGS. 8-10, in which an improved anchor 56 is defined by a single longitudinal cut 58 and a single angled cut 60, which extends from the longitudinal cut 58 to a circumferential edge of a rib of the filter. The angle defined by the longitudinal cut 58 and angled cut 60 may be selected at any desired angle. A tip portion 62 of the anchor 56 is bent in an outward direction, such that a main or base portion 64 of the anchor 56 will act as a spring, tending to gently urge the tip portion 62 of the anchor 56 outward into contact with a vessel wall. FIG. 10 depicts the anchor 56 being pressed inward by a surface or portion of a patient's anatomy (not shown).

Another embodiment of the present invention is shown in FIG. 11, in which an improved anchor 66 is defined by a single longitudinal cut 68 and a single angled cut 70, which extends from the longitudinal cut 68 to a circumferential edge of a rib of the filter. The angle defined by the longitudinal cut 68 and angled cut 70 may be selected at any desired angle. A main or base portion 74 of the anchor 66 is bent in an outward direction, and a tip portion 72 of the anchor 66 is bent to extend essentially parallel to a longitudinal axis of the filter, again to provide a gentle position retention feature. Also, if the filter is a retrievable filter, the configuration of FIG. 11 also provides for easier retrieval of the filter.

Another advantage of this configuration is that it provides two filter elements for intercepting thrombus moving inside the blood vessel. In addition, due to the shape of the filter and ribs, which extend along the internal wall of the blood vessel, the vascular filter provides less possibility for any trauma to a vessel. As the filter sections 28 and 30 have been arranged on either side of the axial ribs defining the central body, a longitudinally symmetrical shape has been obtained. There is no difference whether the vascular filter is placed forward or backward inside the blood vessel. In other words, the proximal and distal ends of the filter are identical and symmetrical. Accordingly, a single pre-loaded catheter system may be used to deploy a filter at a desired site, from either an upstream or downstream direction.

The filter 12 may for example be delivered to the vascular region in the general area of the heart from either a femoral artery access point in the leg, or a jugular artery access point in the neck. Because the filter shown in FIG. 2 is longitudinally symmetrical, the same filter delivery system 10 may be used for either femoral or jugular access, because the opposing sets of anchors 32 will resist downstream migration of the filter, regardless of the longitudinal orientation of the filter.

As has been illustrated, the grid shape of each of the filter sections 28 and 30 is such that each of the ribs 24 is connected to a number of the components of the two filter sections 28 and 30. Furthermore, each of the ribs 24 is connected with both filter sections 28 and 30 on either side. Due to this configuration, even in the unlikely event that one of the ribs 24 or a component of one of the filter sections 28 or 30 may possibly break, the filter 12 will tend not to cause damage nor shift position.

In addition, misalignment or tipping of one or both filter sections 28 and 30 has been effectively avoided due to the more or less tubular shape into which the ribs 24 have been arranged, so that positioning of the vascular filter 12 inside the blood vessel 14 can take place with unprecedented stability and reliability.

In addition, the vascular filter 12 is preferably made as a single piece of a resilient material, such that following deployment from the distal tip 20 of the catheter 16, it will expand and be held in place against the internal wall 26 of the blood vessel 14.

Vascular filters according to the present invention may be made of any suitable material using a variety of methods. One material having the desired characteristics of strength, resilience, flexibility, biocompatibility and endurance is nitinol. Other possible materials include stainless steel and any other material having the desired properties.

Likewise, the manufacturing methods for the filter of the present invention may include providing a tube, and then cutting a pattern into the tube to enable expansion into the desired shape. Various other methods are of course possible, including forming the filter of discrete members and joining or connecting the members, or chemically etching a substrate. The manufacturing methods may include an inflatable or expandable mold, heating or cooling, welding, etc.

In the compressed shape when the vascular filter is inside the catheter, the filter may include cuts extending in the longitudinal direction of the filter between, but not as far as, the ends of the filter. The cuts define strips of material as illustrated in the drawings. These strips correspond to the ribs 24 as illustrated in the drawing Figures. The specific cuts consequently also form the filter elements 28 and 30 on either side of the vascular filter. Those sections of the strips forming the ribs 24 extend in an axial direction and are connected on either side with a filter element 28 or 30, which is formed between the bending lines and the tips on either side of the vascular filter.

Further improvements are illustrated to enhance the resistance of the vascular filter against undesired displacement. In FIGS. 2 and 3 for instance, a rib of a vascular filter according to the present invention has been illustrated, which has been provided with hooking elements or anchors pointing in opposite directions. These hooking elements are sufficiently sized and small enough so that they ensure the proper grip on the internal wall of the blood vessel, but tend not to damage the latter.

Each of the embodiments illustrated in FIGS. 5-11 aims at increasing the reliability of the anchoring of the filter, so that the vascular filter according to the present invention will not shift position. Protrusions such as the projections, anchors or barbs are provided which extend radially outward, due to the elasticity and resilience of the vascular filter and the tendency of the filter to expand, toward contact with the internal wall of a blood vessel. Consequently, resistance against possible displacement under the influence of the flow of fluid or blood through the blood vessel is increased, and as a result the reliability is enhanced.

In the axial view of FIG. 3, the filter sections on either side of the ribs of the vascular filters according to the present invention described above display diamond or polygon shapes. It is also possible to provide vascular filters of which the filter sections display in axial view a star shape, or any other suitable shape, as long as they successfully intercept blood clots or thrombus. An advantage of this feature is that, after passing the first filter section 28 and the tubular section or the elongated body member, a second filter element 30 for intercepting thrombus has been provided. Also, other shapes of the filter sections in axial view are possible, which shapes will occur to those skilled in the field after reading the present description. The shapes of the filter sections in axial view -need not be symmetrical, and may in principle have any suitable appearance.

If it is desirable to be able to remove a vascular filter introduced into a blood vessel at a later stage, referred to as a “retrievable” filter, a vascular filter according to the present invention may be provided with features advantageous to such possible retrieval. For example, the filter may have on one or both ends a hook or loop construction, to extract the vascular filter back into a catheter by means of a cooperating hook, snare or grabbing member.

In addition to the nitinol mentioned so far, many other materials may also be used for manufacturing a vascular filter according to the present invention. By way of alternative, various metals may for instance be used, including stainless steel. In any case, the vascular filter preferably tends to resiliently assume the intended shape hereof after having been ejected from the catheter.

Furthermore, retraction of a vascular filter according to the present invention is mentioned above, which should not limit the scope of the claims attached. As regards the subject of the invention, it is therefore of no consequence whether the filter is placed permanently, in a removable manner, temporarily or otherwise.

It should be understood that an unlimited number of configurations for the present invention could be realized. The foregoing discussion describes merely exemplary embodiments illustrating the principles of the present invention, the scope of which i-s recited in the following claims. Those skilled in the art will readily recognize from the description, claims, and drawings that numerous changes and modifications can be made without departing from the spirit and scope of the invention.

What is claimed is:

1. A vascular filter for placement inside a blood vessel to treat a patient, comprising:
in a radially compressed state, a tubular metal cylinder having a first and second integral cylindrical end collar at a proximal and distal end of the vascular filter; a plurality of ribs arranged in a preselected pattern and extending between the first and second end collar in a direction essentially parallel to a longitudinal axis of the vascular filter;
in a radially expanded deployed state, the ribs tend to resiliently expand outward in radial directions, thereby causing the first and second end collars to move toward each other; each of the ribs tending to spread apart, such that the vascular filter defines a plurality of central polygon shapes arranged adjacently around a central circumference of the vascular filter, each polygon shape being defined by structural members including at least one rib extending essentially parallel to the longitudinal axis of the vascular filter; the vascular filter further defining a first and second end filter portion connecting the central polygons with the end collars, wherein the end filter portions each define a plurality of diamond filter cells; each of the filter cells being formed by a plurality of members; the filter cells all extending along a first and second end cone;
at least one anchor extending outward in a radial direction from at least one of the ribs in the expanded deployed state, which tends to resist longitudinal movement of the filter in a blood vessel, the anchor being defined by one longitudinal cut in a rib essentially parallel to the longitudinal axis, and at least one angled cut in the rib extending from the longitudinal cut to a side of the rib;
such that upon compression and removal of the filter from a blood vessel, the anchor tends to retract radially inward and facilitate removal.

2. The vascular filter as set forth in claim 1, wherein an angle defined by the angled cut and the longitudinal cut is an acute angle.

3. The vascular filter as set forth in claim 1, wherein an angle defined by the angled cut and the longitudinal cut is an obtuse angle.

4. The vascular filter as set forth in claim 1, wherein a tip portion of the anchor is bent in a radially outward direction, such that a remainder of the anchor tends to act as a spring and gently urge the tip portion of the anchor to engage the blood vessel.

5. The vascular filter as set forth in claim 4, wherein a main portion of the anchor is bent in a radially inward direction, and a tip portion of the anchor is bent essentially parallel to the longitudinal axis of the stent.

6. The vascular filter as set forth in claim 1, characterized in that the construction of the filter is such that it has been formed out of one single unitary metal element.

7. The vascular filter as set forth in claim 1, adapted for use in the vena cava.

8. The vascular filter as set forth in claim 1, wherein the polygon shapes are hexagons.

9. The vascular filter as set forth in claim 4, wherein the filter cells have a diamond shape with four sides, defined by a first and second rib each forming a portion of a first and second of the hexagon shapes, and by a first and second conical rib directly connecting an apex of the first and second of the central hexagon shapes with the end collars respectively.
